(19) United States
(12) Patent Application Publication
(10) Pub. No.: US 2026/0311844 A1
(43) Pub. Date: Oct. 8, 2026

(54) METHOD AND SYSTEM FOR MENTAL HEALTH SUPPORT

(71) Applicants: Farzad Ehsani, Sunnyvale, CA (US); Demitrios Master, Sunnyvale, CA (US); Noel Torres, Sunnyvale, CA (US)

(72) Inventors: Farzad Ehsani, Sunnyvale, CA (US); Demitrios Master, Sunnyvale, CA (US); Noel Torres, Sunnyvale, CA (US)

(73) Assignee: Innsightful, Inc., (US)

(21) Appl. No.: 19/078,342

(22) Filed: Mar. 13, 2025

(51) Int. Cl. G16H 20/70

(52) U.S. Cl. CPC G16H 20/70

(57) ABSTRACT

A system, method, and computer program product for automated mental health coaching utilizing a multi-agent architecture powered by large language models. The system employs a hierarchical framework of specialized agents that collaboratively deliver personalized, safe, and effective coaching interactions. A master agent manages overall dialogue flow and maintains conversation history while coordinating with a classifier that selects coaching templates from a template database based on real-time conversational context, ensuring structured yet flexible therapeutic guidance. An interaction agent generates contextually appropriate responses informed by the active template and dialogue history. A verification agent evaluates all generated responses for relevance, methodological consistency, safety, and appropriateness, and a suggestion agent produces alternative responses when verification criteria are not met. A trigger agent continuously monitors dialogue for indicators of self-harm or crisis situations, enabling rapid detection and escalation. Together, these components provide a robust, adaptive platform for automated mental health support.

### CROSS-REFERENCE TO RELATED APPLICATIONS

The present invention claims priority benefit of provisional patent application 63/745, 174 filed on Jan. 14, 2025, and which is incorporated herein by reference in its entirety.

### FIELD OF THE DISCLOSURE

The present invention relates generally to artificial intelligence systems for mental health support, and more particularly to a multi-agent system utilizing large language models for providing automated coaching services. Specifically, the invention concerns a template-based dialogue system with hierarchical verification mechanisms that coordinates multiple specialized agents to deliver personalized mental health coaching while maintaining security and conversation coherence.

### BACKGROUND OF THE INVENTION

Mental health challenges, particularly anxiety and depression, represent a significant and growing healthcare crisis in the United States. As of 2022, approximately 49.6 million adults and 3.7 million youth experienced mental illness, with less than half (41.1%) receiving treatment. The lifetime prevalence of anxiety disorders alone affects roughly 28.8% of the U.S. population, meaning one in four adults has experienced at least one anxiety disorder [1].

The economic impact is equally concerning, with anxiety disorders contributing to an estimated annual cost of $47 billion. More than 75% of this burden stems from indirect costs, including lost productivity, morbidity, mortality, and related expenses [2-3]. Several barriers prevent individuals from accessing necessary mental health care, including high treatment costs, provider shortages, transportation difficulties, and persistent social stigma.

Untreated mental illness carries severe consequences, leading to reduced educational achievement, lower income levels, increased unemployment rates, development of somatic disorders, impaired executive function, and in extreme cases, suicide [4]. Current research emphasizes the critical importance of preventing anxiety from becoming chronic or developing into comorbid conditions, particularly depression.

Real-time stress detection has emerged as a crucial factor in managing and reversing anxiety and depression [5]. Studies demonstrate that detecting anxiety in real-time can prevent negative episodes across various settings, including work, school, and home environments, while simultaneously reducing symptom severity [6].

Contemporary treatment approaches typically combine pharmaceutical interventions with psychotherapy. Cognitive behavioral therapy (CBT) has proven particularly effective by training patients to identify anxiety triggers and modify their response patterns. However, CBT's high cost and required time commitment often limit its accessibility. While pharmaceutical treatments, including antidepressants and anxiolytics, are common, they frequently carry significant side effects and may not be suitable as primary interventions.

The college student population faces particularly acute challenges, with over 50% experiencing anxiety and psychological distress [8]. Primary factors contributing to this crisis include academic performance pressure, future career uncertainty, and general stress about post-graduation life [9-10]. Between 2009 and 2015, college counseling center usage increased by 30-40%, and current data shows depression and anxiety rates among students at 44.2% and 36.6% respectively. These numbers are expected to rise, given that 9.4% of children aged 3-17 years have already received anxiety diagnoses, and 4.4% have been diagnosed with depression [11].

The relationship between anxiety disorders and substance abuse, particularly opioid addiction, presents another critical concern. Research has established that individuals with anxiety disorders show increased susceptibility to drug addiction and abuse [7], highlighting the need for effective, non-pharmaceutical intervention strategies that could yield significant societal benefits.

This complex landscape of mental health challenges, combined with limited access to traditional treatments, creates an urgent need for innovative, accessible, and effective solutions that can provide real-time support while maintaining the quality of care associated with traditional therapeutic approaches.

### Large Language Models

Large Language Models (LLMs) represent one of the most significant advancements in artificial intelligence. These models, trained on massive volumes of text data, excel in generating human-like text, understanding natural language, and performing tasks requiring deep contextual comprehension. The GPT (Generative Pre-trained Transformer) series exemplifies this evolution, with each iteration from GPT-2 through the latest GPT-4.0 (released in May 2024) demonstrating increasing capabilities in scale and functionality.

At the foundation of LLMs lies the Transformer architecture, introduced by Vaswani et al. [12] in 2017, which revolutionized natural language processing by enabling efficient parallel data processing. This marked a significant improvement over previous recurrent neural network (RNN) architectures, which were limited by their sequential processing nature and scalability constraints.

The core innovation of the Transformer architecture is its attention mechanism, particularly self-attention, which enables the model to determine the relative importance of words within a text sequence. This mechanism allows each word to “pay attention” to other words in the sequence, capturing complex contextual relationships and enhancing the model's understanding of word meanings in various contexts.

Modern LLMs leverage this attention mechanism at scale, utilizing hundreds of billions of parameters to capture nuanced linguistic patterns.

These parameters, representing neural connection weights, are fine-tuned during training to optimize the model's performance. The increased parameter count enables better learning of complex language representations, resulting in exceptional performance across various tasks, including text generation, translation, summarization, and question answering [13].

The training process for LLMs demands vast computational resources and extensive datasets. The models' complexity necessitates specialized hardware such as Graphics Processing Units (GPUs) and Tensor Processing Units (TPUs), which handle intensive computations more efficiently than traditional processors. Training typically occurs on distributed computing infrastructures, utilizing multiple GPUs or TPUs in parallel to manage the substantial memory and computational requirements effectively. This sophisticated infrastructure enables the development of increasingly powerful models capable of understanding and generating human-like text with remarkable accuracy and contextual awareness.

### Natural Language Processing

Natural Language Processing (NLP) represents a convergence of computational linguistics and machine learning models, enabling computers and digital devices to understand, generate, and interact with human language. As a fundamental branch of artificial intelligence (AI), NLP underpins applications capable of performing diverse tasks including automatic text translation, response generation to written or spoken commands, user authentication through voice recognition, text synthesis, intention and sentiment analysis, and real-time content generation [14].

Today, NLP technology is ubiquitous in everyday applications, from digital assistants to voice recognition software and customer service chatbots. In the business sector, NLP has demonstrated significant impact by automating processes, enhancing productivity, and streamlining critical operations [15]. However, the inherent complexity and ambiguity of human language present substantial challenges for NLP systems, which must navigate irregularities such as homonyms, sarcasm, idioms, metaphors, and unconventional grammatical structures. While humans naturally interpret these nuances, NLP systems require meticulous programming to process them accurately [16].

To interpret human language effectively, NLP employs various specialized tasks. These include speech recognition (converting spoken language to text), part-of-speech tagging (identifying grammatical roles), word sense disambiguation (determining contextual word meanings), named entity recognition (NER), coreference resolution (identifying when different words refer to the same entity), and sentiment analysis (extracting subjective information from text) [17].

Large Language Models (LLMs), such as GPT-4, represent a specialized subset within the broader NLP field. These models mark a significant advancement through their training on extensive text corpora, enabling them to generate and predict language with unprecedented accuracy.

While LLMs constitute a specific category within NLP, their capability to handle complex language tasks has made them indispensable tools for research and sophisticated application development. The continuous evolution of LLMs drives progress within NLP while expanding the possibilities for human-machine interaction through language. This symbiotic relationship between LLMs and traditional NLP approaches has created new opportunities for advancing both fields simultaneously, leading to more sophisticated and capable language processing systems.

### SUMMARY OF THE INVENTION

The present invention provides systems, methods, and computer program products for automated mental health coaching using a sophisticated multi-agent architecture powered by large language models. In various embodiments, the invention implements a hierarchical system of specialized agents working in concert to deliver personalized, safe, and effective coaching interactions.

In one aspect, the invention provides a system comprising multiple specialized agents, including a master agent that manages overall dialogue flow and maintains conversation history. The master agent works in conjunction with a classifier that selects appropriate coaching templates from a template database based on the ongoing dialogue context. This template-based approach ensures structured and methodical coaching interactions while maintaining flexibility to address various mental health concerns.

The system further comprises an interaction agent responsible for generating contextually appropriate responses based on the active template and dialogue history. To ensure response quality and safety, a verification agent assesses all generated responses against multiple criteria, including relevance to the active template, adherence to coaching methodology, and overall appropriateness. When responses fail to meet these criteria, a suggestion agent generates alternative responses that better align with the coaching objectives and safety requirements.

A particularly important aspect of the invention is the trigger agent, which continuously monitors dialogue for keywords indicating potential self-harm or crisis situations. This safety mechanism enables rapid response to critical situations and appropriate escalation when necessary.

In another aspect, the invention provides a method for automated mental health coaching that begins with receiving user input and maintaining detailed dialogue history. The method includes sophisticated template selection processes where conversation patterns are analyzed and matched against stored templates to identify the most appropriate coaching approach. The selected template guides response generation while maintaining conversation coherence across multiple exchanges.

The method further incorporates comprehensive verification procedures that evaluate responses across multiple dimensions, including relevance, safety, and quality. When responses fail verification, the method provides mechanisms for generating alternative responses that better serve the coaching objectives while maintaining conversation flow.

In yet another aspect, the invention provides a computer program product comprising a non-transitory computer-readable storage medium containing executable code that implements the automated coaching system. The program code enables sophisticated dialogue management, template selection, response generation, and safety verification functions.

A key feature of the invention is its ability to synthesize contextual understanding from dialogue history and adapt coaching strategies based on user engagement patterns. The system tracks conversation progress within templates and identifies appropriate transition points, maintaining continuity as conversation context evolves.

The invention also provides comprehensive progress tracking and assessment capabilities. This includes generating periodic progress assessments, monitoring user engagement metrics, and maintaining records of successful intervention patterns to inform future coaching strategies.

The system's template database represents another crucial component, storing structured interaction steps, predefined response patterns, safety guidelines, and success metrics for verification. These templates ensure consistent and methodical coaching approaches while allowing for personalization based on individual user needs.

Through its various embodiments, the invention addresses the critical need for accessible mental health support while maintaining high standards of safety and effectiveness. The multi-agent architecture, combined with sophisticated template management and verification systems, enables the delivery of personalized coaching support at scale while incorporating necessary safeguards for mental health applications.

### BRIEF DESCRIPTION OF THE DRAWINGS

The FIGURE illustrates a block diagram of an automated mental health coaching system according to an embodiment of the present invention. The system shows the flow of information between multiple specialized agents, beginning with a user request represented by a speech bubble. The diagram depicts a Master Agent that manages overall dialogue flow and maintains both Dialog History and Active Template status. A Classifier component interfaces with a Template Database to select appropriate templates based on dialogue context. An Interaction Agent generates responses using the active template and dialogue history, while a Verifier assesses response quality. A Suggestion Agent component generates alternative responses when necessary. Feedback loops, represented by arrows, show information flow between components, enabling dynamic template updates and system adaptation. The diagram demonstrates the hierarchical relationship between components and the circular nature of the conversation flow, with all components ultimately serving to generate appropriate responses to user inputs while maintaining safety and coherence.

### DETAILED DESCRIPTION OF PREFERRED EMBODIMENTS

The following description of preferred embodiments refers to the accompanying drawings, which illustrate specific embodiments of the invention. Other embodiments having different structures and operations do not depart from the scope of the present invention. The same reference numbers may be used in the drawings and the following description to refer to the same or like parts.

As used herein, the terms “comprising,” “including,” “containing,” “characterized by,” and grammatical equivalents thereof are inclusive or open-ended and do not exclude additional, unrecited elements or method steps, unless otherwise stated. Other than in the operating examples, or where otherwise indicated, all numbers expressing conversation metrics, processing parameters, assessment scores, and so forth used in the specification and claims are to be understood as being modified in all instances by the term “about,” meaning within a reasonable range of the indicated value. The terms “a” and “an” refer to one or more of the elements described, whereas the term “plurality” refers to two or more of the elements described, unless the context clearly indicates otherwise.

The multi-agent mental health coaching system described herein provides novel solutions for automated therapeutic support and continuous dialogue-based coaching interactions. The invention incorporates advanced large language models, sophisticated template-based dialogue management, and hierarchical verification mechanisms, enabling comprehensive mental health coaching while maintaining safety and conversation coherence. The following detailed description, along with the accompanying drawings, provides a comprehensive understanding of the various embodiments and aspects of the invention.

This mental health coaching system addresses the challenges of balancing automated therapeutic support with safety considerations while ensuring timely intervention. It offers a versatile solution that combines advanced natural language processing with structured coaching templates, potentially improving mental health outcomes through accessible and continuous support. The invention's ability to provide automated coaching capabilities while maintaining conversation quality and safety represents a significant advancement in mental health technology.

The present invention provides a comprehensive multi-agent system for automated mental health coaching utilizing large language models (LLMs) and sophisticated dialogue management techniques. The system combines advanced natural language processing capabilities with structured coaching methodologies to deliver personalized therapeutic support while maintaining safety and conversation coherence.

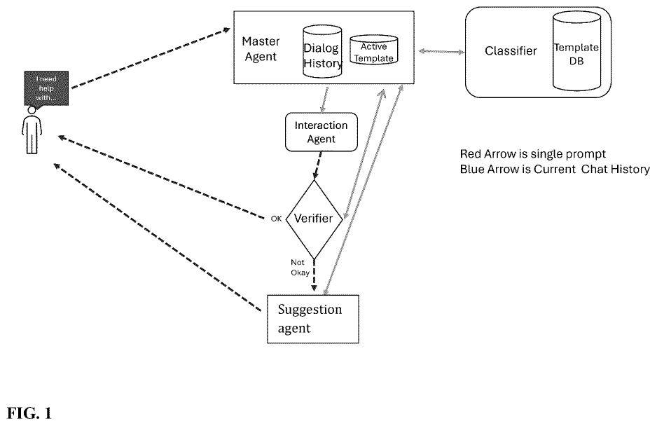

Referring to FIG. 1, the system comprises several interconnected components that work together to provide automated mental health coaching. At the core of the system is a Master Agent that manages overall dialogue flow and coordinates interactions between various specialized agents. The Master Agent serves as the central coordinator of the system, maintaining two critical elements: the Dialog History and Active Template status. The Dialog History represents a comprehensive record of all interactions within a coaching session, enabling the system to maintain context and coherence across multiple exchanges. The Active Template status tracks the currently selected coaching approach, which guides the generation of responses and ensures structured therapeutic interactions.

The Master Agent implements sophisticated dialogue management through a dialogue history storage module that maintains comprehensive conversation records, a template tracking module that monitors the current active template, and a feedback processing module that enables dynamic template updates based on conversation flow. These components work in concert with coordination mechanisms for managing information flow between other system components.

The system maintains a Template Database containing a plurality of coaching interaction templates. Each template comprises structured interaction steps defining the progression of coaching conversations, predefined response patterns appropriate for specific therapeutic contexts, safety guidelines ensuring appropriate handling of sensitive topics, and success metrics for verifying response quality and effectiveness. The Classifier component interfaces with the Template Database to select appropriate templates based on dialogue context. It employs sophisticated analysis techniques to analyze recent dialogue history, match conversation patterns against template parameters, calculate matching scores for potential templates, select the most appropriate template based on matching scores, and update template selection as conversation context evolves.

The Classifier plays a crucial role in maintaining coaching effectiveness by ensuring that the active template aligns with the current therapeutic needs of the user. When conversation patterns indicate a need for a different approach, the Classifier signals the Master Agent to update the Active Template accordingly. The Interaction Agent generates responses based on multiple inputs, including the current Active Template providing structure and guidance, complete dialogue history enabling context-aware responses, user input patterns informing response style and tone, and coaching objectives defined within the template.

The Verification Agent serves as a critical safety and quality control mechanism within the system. When the Interaction Agent generates a potential response, the Verification Agent assesses it across multiple dimensions to ensure it meets the system's standards for therapeutic value and safety.

Preferably, the system implements a response evaluation process that ensures the quality and therapeutic value of all coaching interactions. Each generated response undergoes comprehensive assessment across multiple dimensions, including its relevance to the active template, linguistic coherence, emotional appropriateness, and context alignment. The Verification Agent examines how well responses align with predefined coaching objectives while analyzing sentence structure, grammar, and logical flow. Special attention is given to detecting empathetic and supportive tones, ensuring responses maintain appropriate emotional resonance while verifying consistency with prior user inputs. When responses fail to meet these criteria, the Suggestion Agent engages in a refined optimization process. This includes adjusting response phrasing to enhance clarity and engagement, re-ranking alternative responses to select the most contextually relevant options, and incorporating prompt tuning techniques that modify internal instructions to generate more suitable responses. The Verification Agent employs probabilistic scoring techniques to ensure final responses meet predefined confidence thresholds for relevance and quality. This multi-step refinement process maintains conversation coherence while enhancing user engagement and adhering to therapeutic best practices, representing a critical aspect of the system's ability to deliver effective automated coaching support.

This verification process examines the response's relevance to the active template, adherence to coaching methodology steps, overall safety considerations, and quality metrics such as coherence and appropriateness. The Verification Agent's assessment is based on predefined criteria stored within each template, ensuring that responses align with therapeutic objectives while maintaining consistent quality standards.

In cases where the Verification Agent identifies deficiencies in a generated response, the system engages the Suggestion Agent. The Suggestion Agent has access to the complete dialogue history, active template, and the original generated response, enabling it to produce alternative responses that better align with therapeutic goals and safety requirements. This component ensures that even when initial responses fail verification, the system can maintain productive therapeutic interactions without compromising quality or safety.

A particularly crucial component of the system is the Trigger Agent, which continuously monitors all dialogue for predefined keywords and patterns that might indicate potential self-harm or crisis situations. The Trigger Agent maintains a comprehensive database of crisis-indicating keywords and key phrases as well as particular scenarios and performs real-time analysis of user inputs. When triggers are detected, the system can immediately escalate to appropriate crisis response protocols. All trigger events are logged for system improvement and future reference, contributing to the ongoing refinement of the system's safety mechanisms.

The system implements sophisticated feedback loops throughout its architecture, enabling continuous refinement and adaptation of coaching strategies. These feedback mechanisms ensure that information flows back to the Master Agent and other components as needed, allowing for dynamic updates to the active template and coaching approach. The feedback system enables the Classifier to intervene and modify the Active Template when conversation patterns indicate a need for adjustment, ensuring that the coaching interaction remains responsive to changing user needs.

The system incorporates an advanced automated refinement mechanism that continuously enhances the quality and effectiveness of coaching interactions. This mechanism implements real-time evaluation of past responses, enabling dynamic adjustments based on observed user interaction trends and patterns. Through sophisticated adaptive learning techniques, the system optimizes prompt engineering and refines conversational outputs to better serve therapeutic objectives. A probabilistic response scoring system dynamically adjusts LLM-generated outputs based on historical effectiveness metrics, ensuring continuous improvement in response quality. The mechanism also features feedback-driven template evolution, where frequently utilized coaching patterns undergo automatic fine-tuning to increase their relevance and effectiveness over time. This comprehensive learning-based improvement system operates continuously in the background, analyzing interaction patterns and adjusting response parameters to maintain optimal therapeutic value. By incorporating these sophisticated refinement capabilities, the system ensures that coaching interactions consistently align with mental health best practices while adapting to emerging user needs and interaction patterns, representing a significant advancement in automated therapeutic support technology.

Progress tracking and assessment capabilities are integrated throughout the system. The computer program product implementing these features enables the generation of periodic progress assessments, tracking of user engagement metrics, and maintenance of records documenting successful intervention patterns. This data helps inform future coaching strategies and enables continuous improvement of the system's effectiveness. The tracking system monitors various metrics including conversation coherence, user engagement levels, successful template transitions, and overall therapeutic progress.

Template transitions represent a particularly sophisticated aspect of the system's operation. The program code enables tracking of conversation progress within templates, identification of appropriate transition points, and selection of new templates when conversation context changes. During these transitions, the system maintains continuity by preserving relevant context while adapting to new therapeutic approaches as needed. This capability ensures smooth progression through different coaching phases while maintaining coherent and effective therapeutic interactions.

The system's security features extend beyond basic safety checks to include comprehensive content verification and crisis response protocols. All generated responses undergo multiple layers of verification, ensuring not only technical accuracy but also therapeutic appropriateness. The system maintains detailed logs of all interactions, enabling thorough analysis of coaching effectiveness while preserving user privacy and confidentiality. These security measures are implemented at multiple levels, from initial response generation through final delivery to the user.

In terms of implementation, the system utilizes advanced natural language processing techniques to maintain conversation coherence across multiple exchanges. This includes sophisticated context tracking mechanisms that enable the system to reference and build upon previous interactions while maintaining therapeutic relevance. The Interaction Agent's response generation capabilities are enhanced by its ability to incorporate historical context from previous exchanges, ensuring that each response builds meaningfully upon the established therapeutic relationship.

The Template Database represents a crucial knowledge repository within the system, storing not only basic conversation patterns but also sophisticated therapeutic strategies aligned with established coaching methodologies. Templates are structured to guide conversations through various therapeutic phases while maintaining flexibility to adapt to individual user needs. Each template contains specific guidance for handling different therapeutic scenarios, including detailed instructions for response generation and verification criteria.

Communication between system components is managed through a sophisticated message passing architecture that ensures reliable information flow while maintaining system stability. The Master Agent coordinates these communications, ensuring that each component receives the information necessary for its operation while maintaining overall system coherence. This coordination extends to managing the timing and sequencing of interactions between components, ensuring smooth operation even during complex therapeutic exchanges.

The verification process implemented by the Verification Agent includes multiple stages of assessment. Initial verification focuses on basic safety and appropriateness, followed by more detailed analysis of therapeutic value and alignment with coaching objectives. The system maintains detailed verification metrics that help track the effectiveness of different coaching strategies while identifying areas for improvement. When responses require modification, the Suggestion Agent employs sophisticated analysis techniques to identify specific areas for improvement and generate more appropriate alternatives.

User engagement tracking is implemented through various metrics that help the system adapt its coaching approach based on user responses. The system monitors factors such as response patterns, engagement levels, and progress toward therapeutic goals, using this information to refine its coaching strategies. This adaptive capability enables the system to provide increasingly personalized and effective support over time, learning from successful interaction patterns to improve future coaching sessions.

Crisis response capabilities are integrated throughout the system architecture, with the Trigger Agent maintaining constant vigilance for potential warning signs. When crisis indicators are detected, the system can rapidly shift to appropriate response protocols, ensuring timely and appropriate intervention. These crisis response mechanisms are carefully designed to provide appropriate support while maintaining user safety and ensuring proper escalation when necessary.

The system's feedback mechanisms enable continuous improvement through sophisticated learning algorithms that analyze interaction patterns and outcomes. This includes tracking successful coaching strategies, identifying effective template transitions, and refining verification criteria based on observed results. The feedback system helps optimize the selection and application of coaching templates while improving the overall effectiveness of therapeutic interactions.

Implementation of the template selection process involves sophisticated pattern matching algorithms that analyze conversation context to identify the most appropriate coaching approach. The Classifier employs multiple analysis techniques to evaluate potential templates, including assessment of conversation tone, topic relevance, and therapeutic alignment. Template selection criteria include factors such as conversation history, user engagement patterns, and progress toward therapeutic goals, ensuring that selected templates provide appropriate guidance for each stage of the coaching interaction.

The system's response generation capabilities leverage advanced natural language processing techniques to produce contextually appropriate and therapeutically valuable responses. The Interaction Agent considers multiple factors when generating responses, including the active template's guidance, historical conversation context, and specific therapeutic objectives. Generated responses are designed to maintain conversation coherence while advancing therapeutic goals, with careful attention to maintaining appropriate tone and style throughout the interaction.

Security and privacy considerations are implemented at multiple levels throughout the system. All user interactions are processed with appropriate privacy protections, and sensitive information is handled according to established security protocols. The system implements robust data protection measures while maintaining the ability to track and analyze coaching effectiveness. These security measures extend to all aspects of system operation, from initial user input through response generation and delivery.

Progress tracking capabilities include sophisticated metrics for assessing therapeutic effectiveness and user engagement. The system maintains detailed records of successful intervention patterns, enabling continuous refinement of coaching strategies. Progress assessment includes evaluation of multiple factors including response patterns, engagement levels, and advancement toward therapeutic objectives. This comprehensive tracking enables the system to adapt its coaching approach based on observed effectiveness while maintaining therapeutic consistency.

The system's crisis response capabilities include detailed protocols for handling potentially dangerous situations. When the Trigger Agent identifies crisis indicators, the system can rapidly implement appropriate response measures while maintaining user safety. Crisis response protocols include specific guidance for different types of situations, ensuring appropriate handling of various crisis scenarios while maintaining therapeutic support.

The feedback mechanisms implemented throughout the system enable continuous refinement of coaching strategies. This includes analysis of successful interaction patterns, evaluation of template effectiveness, and assessment of verification criteria. The feedback system helps optimize coaching effectiveness while maintaining appropriate therapeutic standards and safety measures. These mechanisms enable the system to learn from successful interactions while identifying areas for improvement in coaching delivery.

Implementation of the verification process includes multiple stages of assessment to ensure response quality and safety. The Verification Agent employs sophisticated analysis techniques to evaluate responses across multiple dimensions, including therapeutic value, safety considerations, and alignment with coaching objectives. When responses require modification, the Suggestion Agent generates alternatives that maintain therapeutic value while addressing identified concerns.

The operational aspects of the automated mental health coaching system encompass several key functional areas. During typical operation, the system begins by receiving user input through a text-based interface. This input is immediately processed by the Master Agent, which maintains the ongoing dialogue history and coordinates the system's response generation process. The Master Agent's operational role includes ensuring conversational continuity and managing the flow of information between system components.

When processing a new user input, the Classifier operationally interfaces with the Template Database to evaluate and select appropriate coaching templates. This selection process occurs in real-time, with the Classifier analyzing recent dialogue history to identify the most appropriate therapeutic approach. Once a template is selected, the Master Agent updates the Active Template status, ensuring all system components align with the current coaching strategy.

The Interaction Agent operates continuously to generate responses based on the active template and dialogue context. This process involves analyzing user input, consulting the dialogue history, and applying template-specific guidance to produce appropriate therapeutic responses. The generation process incorporates multiple contextual factors to ensure responses maintain conversation coherence while advancing therapeutic objectives.

During operation, the Verification Agent provides continuous quality control by assessing all generated responses before they reach the user. This verification process operates in real-time, evaluating responses across multiple criteria including safety, therapeutic value, and alignment with coaching objectives. When responses fail verification, the Suggestion Agent automatically engages to generate alternative responses that better meet system requirements.

The Trigger Agent operates in parallel with other system components, continuously monitoring all dialogue for potential crisis indicators. This monitoring occurs in real-time, enabling immediate response to potential safety concerns. When triggers are detected, the system can rapidly implement appropriate crisis response protocols while maintaining therapeutic support.

Feedback mechanisms operate continuously throughout system operation, enabling dynamic updates to coaching strategies based on observed effectiveness. This includes real-time updates to template selection, response generation parameters, and verification criteria. The feedback system enables continuous refinement of coaching effectiveness while maintaining appropriate therapeutic standards.

Progress tracking operates alongside other system functions, maintaining detailed records of therapeutic interactions and outcomes. This tracking includes continuous assessment of user engagement, response patterns, and progress toward therapeutic objectives. The tracking system enables ongoing evaluation of coaching effectiveness while informing adjustments to therapeutic strategies.

The system's operational success relies heavily on the coordination between its various components and agents. Template transitions occur smoothly during operation, with the system maintaining therapeutic continuity while adapting to changing user needs. The Master Agent ensures proper synchronization of all components during these transitions, maintaining conversation coherence throughout the coaching process.

Security measures operate continuously during system operation, protecting user privacy while enabling effective therapeutic support. All interactions are processed according to established security protocols, with appropriate safeguards maintaining data protection throughout the coaching process. These security measures ensure safe and confidential operation while enabling effective therapeutic support.

The system's ability to maintain effective therapeutic interactions depends on sophisticated coordination between multiple specialized agents. During operation, these agents work in concert to provide structured, safe, and effective coaching support. The Master Agent's coordination ensures smooth operation across all system components, maintaining therapeutic effectiveness while adapting to user needs.

Through its various operational aspects, the system provides accessible, continuous mental health support while maintaining high standards of safety and effectiveness. The multi-agent architecture, combined with sophisticated template management and verification systems, enables the delivery of personalized coaching support at scale. This operational framework represents a significant advancement in automated mental health support technology, addressing the critical need for accessible therapeutic resources while maintaining appropriate safety and quality standards.

The implementation and operation of this system demonstrates the potential for automated coaching systems to provide valuable mental health support while maintaining necessary safety and quality controls. Through careful coordination of multiple specialized agents and sophisticated dialogue management techniques, the system offers a promising approach to addressing the growing need for accessible mental health resources.

### INDUSTRIAL APPLICATION

The automated mental health coaching system described herein has direct industrial applications in healthcare, educational institutions, and corporate wellness programs. The system can be deployed as a scalable solution for providing 24/7 mental health support, particularly in settings where traditional therapeutic resources are limited or costly. It is especially valuable for universities managing large student populations, corporations implementing employee wellness programs, and healthcare providers seeking to extend mental health services. The system's ability to provide consistent, safe, and personalized coaching support while maintaining quality standards makes it particularly suitable for large-scale implementation across various institutional settings.

### REFERENCES

[1]-[11] not from my text
[12] Vaswani, A., Shazeer, N., Parmar, N., Uszkoreit, J., Jones, L., Gomez, A. N., Kaiser, L., and Polosukhin, I. (2023). Attention is all you need.
[13] Ray, P. P. (2023). Chatgpt: A comprehensive review on background, applications, key challenges, bias, ethics, limitations and future scope. Internet of Things and Cyber-Physical Systems, 3:121-154.
[14] Opitz, J., Wein, S., and Schneider, N. (2024). Natural language processing relies on linguistics.
[15] Ive, J. (2022). Natural Language Processing: A Machine Learning Perspective by Yue Zhang and Zhiyang Teng. Computational Linguistics, 48 (1): 233-235.
[16] Khurana, D., Koli, A., Khatter, K., and Singh, S. (2023). Natural language processing: state of the art, current trends and challenges. Multimedia Tools and Applications, 82(3): 3713-3744.
[17] Mitkov, R. (2022). The Oxford Handbook of Computational Linguistics. Oxford University Press.
[18] Zhang, Y., Sun, S., Galley, M., Chen, Y.-C., Brockett, C., Gao, X., Gao, J., Liu, J., and Dolan, B. (2020). Dialogpt: Large-scale generative pretraining for conversational response generation. In Proceedings of the 58th Annual Meeting of the Association for Computational Linguistics, pages 270-278.
[19] Pichai, S. and Hassabis, D. (2024). Our next-generation model: Gemini 1.5.
   https://blog. google/technology/ai/ our-next-generation-model-gemini-1-5/. Accessed: 2024-09-04.
[20] Gao, J., Galley, M., and Li, L. (2019). Neural approaches to conversational ai. Foundations and Trends in Information Retrieval, 13(2-3):127-298.
[21] Li, J., Monroe, W., Ritter, A., Galley, M., Gao, J., and Jurafsky, D. (2017). Deep reinforcement learning for dialogue generation. In Proceedings of the 2017 Conference on Empirical Methods in Natural Language Processing, pages 1192-1202.
[22] Möller, S., Englert, R., Engelbrecht, K.-P., Hafner, V., Jameson, A., Oulasvirta, A., Raake, A., and Reithinger, N. (2006). Memo: Towards automatic usability evaluation of spoken dialogue services by user error simulations. In Proceedings of Interspeech 2006, pages 1740-1743.
[23] Mehri, S. and Eskenazi, M. (2020). Unsupervised evaluation of interactive dialog with dialogpt.
[24] Dziri, N., Kamalloo, E., Mathewson, K. W., and Zaiane, 0. (2020). Evaluating coherence in dialogue systems using entailment.
[25] Guingrich, R. E. and Graziano, M. S. A. (2024). Chatbots as social companions: How people perceive consciousness, human likeness, and social health benefits in machines. arXiv preprint arXiv: 2311.10599. Presented at ASSC '26 at NYU.
[26] Jones, C. R. and Bergen, B. K. (2024). People cannot distinguish gpt-4 from a human in a turing test.
[27] Bergmann, D. (2023). ¿qué es el aprendizaje por refuerzo a partir de la retroal imentación humana (rlhf)?
[28] Wei, J., Wang, X., Schuurmans, D., Bosma, M., Ichter, B., Xia, F., Chi, E., Le, Q., and Zhou, D. (2023). Chain-of-thought prompting elicits reasoning in large language models.
[29] Madaan, A., Tandon, N., Gupta, P., Hallinan, S., Gao, L., Wiegreffe, S., Alon, U., Dziri, N., Prabhumoye, S., Yang, Y., Welleck, S., Majumder, B. P., Gupta, S., Yazdanbakhsh, A., and Clark, P. (2023). Selfrefine: Iterative refinement with self-feedback.
[30] Shinn, N., Cassano, F., Berman, E., Gopinath, A., Narasimhan, K., and Yao, S. (2023). Reflexion: Language agents with verbal reinforcement learning.
[31] Yang, Z., Li, L., Wang, J., Lin, K., Azarnasab, E., Ahmed, F., Liu, Z., Liu, C., Zeng, M., and Wang, L. (2023). Mm-react: Prompting chatgpt for multimodal reasoning and action.
[32] Bluckert, P. (2005). The similarities and differences between coaching and therapy. Industrial and Commercial Training, 37:91-96.
[33] Bachkirova, T. (2017). Developing a knowledge base of coaching: Questions to explore. In Bachkirova, T., Spence, G., and Drake, D., editors, The SAGE handbook of coaching, pages 23-41. Sage Publications, Inc.
[34] Yordanova, S., Dineva, S., and Obrenovic, B. (2022). Emotion regulation at work employees and leaders' perspectives. International Journal of Innovation and Economic Development, 8.
[35] Segers, J. and Ilke, I. (2012). Exploring supportive and developmental career management through business strategies and coaching. Human Resource Management, 51.
[36] Berg, M. and Karlsen, J. (2007). Mental models in project management coaching. Engineering Management Journal; EMJ, 19:3-13.
[37] Liang, K.-H., Shi, W., Oh, Y., Wang, H.-C., Zhang, J., and Yu, Z. (2022). Dialoging resonance: How users perceive, reciprocate and react to chatbot's self-disclosure in conversational recommendations.
[38] Goel, A., Daheim, N., and Gurevych, I. (2024). Socratic reasoning improves positive text rewriting.
[39] Sharma, A., Rushton, K., Lin, I. W., Wadden, D., Lucas, K. G., Miner, A. S., Nguyen, T., and Althoff, T. (2023). Cognitive reframing of negative thoughts through human-language model interaction.
[40] Abravanel, M. and Gavin, J. (2017). Exploring the evolution of coaching through the lens of innovation. International Journal of Evidence Based Coaching and Mentoring, 15(1): 24-41.
[41] Jelodari, M., Amirhosseini, M. H., and Giraldez-Hayes, A. (2023). An ai powered system to enhance self-reflection practice in coaching. Cognitive Computation and Systems, 5:n/a-n/a.
[42] Sun, Y. and Martin, D. (2023). Protect function in cybersecurity: Llms as proactive tools against harmful content. arXiv preprint arXiv: 2402. 00891. Last accessed: Sep. 10, 2024.

1. A system for providing automated mental health coaching, comprising:
a master agent configured to manage dialogue flow and maintain dialogue history;
a template database storing coaching interaction templates;
a classifier configured to select active templates from the template database based on dialogue context;
an interaction agent configured to generate responses based on active templates and dialogue history;
a verification agent configured to evaluate generated responses across multiple criteria including relevance, linguistic coherence, emotional appropriateness, and context alignment;
a suggestion agent configured to refine responses through adaptive learning techniques when responses fail verification;
a trigger agent configured to monitor dialogue for crisis indicators;
an automated refinement mechanism configured to:
evaluate past responses in real-time;
optimize prompt engineering through adaptive learning;
implement probabilistic response scoring; and
evolve templates based on interaction feedback; and
feedback loops configured to enable continuous system adaptation and improvement based on interaction patterns and outcomes.

2. The system of claim 1, wherein the master agent comprises a dialogue history storage module, a template tracking module, and a feedback processing module that enables dynamic template updates based on conversation flow.

3. The system of claim 1, wherein each coaching interaction template in the template database comprises structured interaction steps, predefined response patterns, safety guidelines, and success metrics for verifying response quality and effectiveness.

4. The system of claim 1, wherein the verification agent is configured to assess responses based on predefined criteria stored within each template to ensure responses align with therapeutic objectives while maintaining consistent quality standards.

5. The system of claim 1, wherein the trigger agent maintains a database of crisis-indicating keywords and performs real-time analysis of user inputs to detect potential self-harm or crisis situations.

6. The system of claim 1, wherein the system implements progress tracking capabilities that generate periodic assessments, monitor user engagement metrics, and maintain records of successful intervention patterns.

7. A method for providing automated mental health coaching, comprising:
receiving user input through a text-based interface;
maintaining dialogue history of interactions;
selecting an appropriate coaching template from a template database based on dialogue context ;
generating responses based on the selected template and dialogue history;
verifying generated responses across multiple criteria including relevance, linguistic coherence, emotional appropriateness, and context alignment ;
refining responses through adaptive learning techniques when responses fail verification;
monitoring dialogue for crisis indicators;
implementing an automated refinement mechanism that evaluates past responses, optimizes prompt engineering, and evolves templates based on interaction feedback; and
enabling continuous system adaptation and improvement through feedback loops based on interaction patterns and outcomes.

8. The method of claim 7, wherein selecting an appropriate coaching template comprises analyzing recent dialogue history, matching conversation patterns against template parameters, calculating matching scores for potential templates, and selecting the most appropriate template based on matching scores.

9. The method of claim 7, wherein generating responses comprises considering the active template, complete dialogue history, user input patterns, and coaching objectives defined within the template.

10. The method of claim 7, wherein verifying generated responses comprises assessing relevance to the active template, adherence to coaching methodology steps, safety considerations, and quality metrics including coherence and appropriateness.

11. The method of claim 7, wherein monitoring dialogue for crisis indicators comprises analyzing user inputs for predefined keywords and patterns indicating potential self-harm or crisis situations.

12. The method of claim 7, further comprising tracking progress by generating periodic assessments, monitoring user engagement metrics, and maintaining records of successful intervention patterns.

13. A non-transitory computer-readable storage medium containing executable code for providing automated mental health coaching, the executable code when executed by a processor causes the processor to:
receive user input through a text-based interface;
maintain dialogue history of interactions;
select an appropriate coaching template from a template database based on dialogue context;
generate responses based on the selected template and dialogue history;
verify generated responses across multiple criteria including relevance, linguistic coherence, emotional appropriateness, and context alignment;
refine responses through adaptive learning techniques when responses fail verification;
monitor dialogue for crisis indicators;
implement an automated refinement mechanism that evaluates past responses, optimizes prompt engineering, and evolves templates based on interaction feedback; and
enable continuous system adaptation and improvement through feedback loops based on interaction patterns and outcomes.

14. The non-transitory computer-readable storage medium of claim 13, wherein the executable code for selecting an appropriate coaching template comprises code for analyzing recent dialogue history, matching conversation patterns against template parameters, calculating matching scores for potential templates, and selecting the most appropriate template based on matching scores.

15. The non-transitory computer-readable storage medium of claim 13, wherein the executable code for generating responses comprises code for considering the active template, complete dialogue history, user input patterns, and coaching objectives defined within the template.

16. The non-transitory computer-readable storage medium of claim 13, wherein the executable code for verifying generated responses comprises code for assessing relevance to the active template, adherence to coaching methodology steps, safety considerations, and quality metrics including coherence and appropriateness.

17. The non-transitory computer-readable storage medium of claim 13, wherein the executable code for monitoring dialogue for crisis indicators comprises code for analyzing user inputs for predefined keywords and patterns indicating potential self-harm or crisis situations.

18. The non-transitory computer-readable storage medium of claim 13, wherein the executable code further comprises code for tracking progress by generating periodic assessments, monitoring user engagement metrics, and maintaining records of successful intervention patterns.

19. The non-transitory computer-readable storage medium of claim 13, wherein the executable code for implementing an automated refinement mechanism comprises code for dynamically adjusting response generation parameters based on historical effectiveness metrics.
